(19) United States
(12) Patent Application Publication
(10) Pub. No.: US 2011/0318778 A1
(43) Pub. Date: Dec. 29, 2011

(54) Organic Substrate Treatment System

(76) Inventors: GERT BJØRN PETERSEN, Haarby (DK); Carsten Moberg, Brenderup (DK); Karsten Hammer Langgaard, Holstebro (DK)

(21) Appl. No.: 12/825,508

(22) Filed: Jun. 29, 2010

(51) Int. Cl. C12P 1/00; C12M 1/00

(52) U.S. Cl. 435/41; 435/303.2; 435/299.1

(57) ABSTRACT

The present invention is directed to an organic substrate treatment system that converts organic substrate and other organic materials to nutrient rich effluent and biogas. Using sequential degradation of the organic materials, biogas and effluent is efficiently produced with relatively short digestion times. Additives are added to the organic materials prior to anaerobic digestion to enhance biogas production. A desulfurizer may be used to clean the raw biogas so that it is suitable for use with gas engines and boilers.

### BACKGROUND OF THE INVENTION

The present invention is directed to a method and apparatus for recovering biogas from organic substrate.

Farmers have long recognized the benefits of composted animal and organic substrate as a natural fertilizer. In a typical manure treatment system, manure is stored in storage tanks or lagoons and upon such storage, anaerobic digestion breaks down the substrate ultimately to methane, carbon dioxide, and a nutrient rich sludge. During the degradation process, however, intermediate compounds are produced which can be volatile and a source of unpleasant odors. As such, many farmers, as well as other firms that produce large amounts of organic substrate, such as milk producers, cheese makers, breweries, and distilleries, and wastewater and sewage treatment facilities, are turning to well-controlled anaerobic digesters to more effectively break down the organic substrate. With a well-controlled and balanced anaerobic digester, the growth of microorganisms can be promoted to break down the substrate without releasing the many compounds that produce the aforementioned unpleasant odors.

In addition to the odor reducing benefits, a well-controlled anaerobic digester can be used to effectively promote and capture biogas that is a byproduct of the digestion process. This biogas can then be burned onsite for heat energy or converted to electricity. Alternately, the biogas can be stored, shipped, and ultimately sold as a secondary revenue source.

In general, anaerobic digestion is a series of processes by which microorganisms break down the organic substrate in the absence of oxygen to provide biogas and a digestate that can be used as fertilizer. Typical anaerobic digestion begins with bacterial hydrolysis of the organic substrate in order to break down insoluble organic polymers such as carbohydrates and make them available for other bacteria. Acidogenic bacteria then convert the sugars and amino acids into carbon dioxide, hydrogen, ammonia, and organic acids. Acetogenic bacteria then convert these resulting organic acids into acetic acid, along with additional ammonia, hydrogen, and carbon dioxide. Finally, methanogens convert these products to methane and carbon dioxide.

A conventional anaerobic digester generally consists of a reactor tank loaded with media for biofilm formation. The internal media can take many fauns. U.S. Pat. No. 4,183,809 describes a digester in which anaerobic microorganisms are suspended in a liquid medium. The internal media can also take the form of particulate matter, such as sand, wood chips, or gravel, contained with the reactor tank, as described in U.S. Pat. Nos. 4,366,059, 4,780,198, and 5,419,833. The particulate matter provides a surface area on which the biofilm may grow. In a similar fashion, some anaerobic digestion systems have reactors loaded with corrugated tubes that provide a relatively large surface area for biofilm formation.

Conventional digesters are generally one of three types: continuous stirred tank reactor (CSTR), anaerobic digester ultra filtered (ADUF), and fixed film tubular plug flow (FFTP). While there are differences between the types of digesters, the same biological process takes place in each digester. Hydrolysing bacteria degradates complex organic molecules (biomass) to various compounds including volatile fatty acids, alcohols, carbon dioxide, and hydrogen gas. Bacteria then convert these products to a methane-based biogas and an effluent (“digestate”) that can be used as fertilizer.

CSTR and ADUF digesters use mobile bacteria within the reactor tank whereas FFTP digesters use immobile or fixed bacteria. These differences in bacteria result in the degradation time with each reactor type varying significantly, and these degradation times determine the volume of the digester that is needed for a particular application. For example, conventional CSTR digesters typically require 20 days to break down biomass to effluent and biogas. Thus, for a conventional CSTR digester or plant to process 200 tons of biomass per day, the digester size would need to be over 1 million gallons (200 tons×20=1,060,000 gal.). Conventional ADUF digesters typically have a degradation time of 6-10 days, and thus, to process 200 tons of biomass per day, the digester would need to be approximately 500,000 gallons. A FFTP digester can process biomass in as little as four days allowing for a significantly smaller digester tank.

Regardless of the type of digester process and internal media, convention anaerobic digestion systems generally utilize a single reactor tank for the degradation process. Animal waste (biomass) is fed to the reactor tank and passes through the internal media which causes the production of methane that is collected via a biogas outlet and the formation of a sediment or effluent layer at the bottom of the tank. The effluent, once removed from the tank, can then be used as nutrient rich fertilizer.

For CSTR and ADUF digesters, this single tank can be quite large and thus require a large physical footprint. FFTP digesters, since they generally process the biomass more quickly, allow for a smaller footprint. However, one of the concerns with FFTP digesters, and CSTR and ADUF digesters to a limited extent, is achieving a proper biological balance within the digester tank. To effectively and efficiently produce biogas, there must be a balance between the bacteria that breaks down the organic substrate (“the degradating bacteria”) and bacteria that converts the degradating bacteria to methane (“the methane producing bacteria”). It is generally known that degradating bacteria, which converts the organic parts of the biomass into volatile fatty acids, alcohols, carbon dioxide and hydrogen gas, work and replicate quickly under high and low pH conditions as well as high and low temperatures. Contrastingly, methane producing bacteria, which convert the “degradator” products into biogas (principally, methane), work and replicate quite slowly and only under well-defined pH and temperature conditions. As a result, if the digester tank is not well balanced, the organic substrate may be decomposed quite quickly but biogas formation can be little, if any. For large digester tanks, providing the necessary balancing can be quite challenging.

Furthermore, many anaerobic digesters are fed organic substrate in a flushed biomass. That is, the organic substrate is flushed from a storage location, e.g., barn, using water. This flushed water is then input to the digester tank for degradation of the organic substrate and the formation of biogas. While effective in removing the organic substrate from the barn, for example, the introduction of water can negatively impact the degradation and biogas formation processes. In short, the water is an energy-free compound and thus its introduction to the digester tank does not result in any energy benefits. In fact, unless the water is drained from the digester tank, the addition of water to the organic substrate adds to the volume requirements of the digester tank or reduces the amount of organic substrate that can actually be input to the digester tank.

Another drawback of conventional waste treatment systems is the post-collection treatment of biogas. Raw biogas contains methane, carbon dioxide, hydrogen sulphide, ammonia, nitrogen, and water vapor. Methane is an odorless, colorless non-poisonous gas which is lighter than atmospheric air and is combustible/inflammable. Hydrogen sulphide is a heavy gas, which is corrosive when burnt as a gas and converts to sulfuric acid when it is dissolved in water. If not removed from the biogas, the combustion of the biogas causes significant sulfur dioxide exhaust emissions. Raw biogas also contains siloxanes. During the combustion of biogas that contains siloxanes, silicon dioxide is formed as crystals in lubricating oil and scaling in cylinders, on valves, pistons, and the like. All of which can greatly increase wear and tear on gas engines. The amounts of the above constituents largely depend on the type of organic material that is digested for biogas formation. For example, if the organic material has a larger protein content, the amount of ammonia and hydrogen in the raw biogas will be higher.

The removal of the hydrogen sulphide is the primary objective of a biogas cleansing process. A number of methods have been developed for the removal of sulfur from biogas. Generally speaking, conventional sulfur removal processes are inefficient or require the uneconomically high consumption of chemicals.

### SUMMARY OF THE INVENTION

The present invention generally provides a system for the treatment and utilization of organic substrate streams, including the cleansing of raw biogas produced from anaerobic digestion of organic substrate. Through a well-controlled refining process, the organic substrate streams are converted to valuable fractions, such as energy in the form of biogas and nutrient-rich compounds containing nitrogen, phosphorous, and calcium concentrates.

The system includes a pre-treatment system that prepares the raw, organic substrate prior to its injection to a digestion system. During the pre-treatment process, the organic substrate is transformed into a thickened slurry or substrate. Thickening the substrate avoids the injection or pumping of large amounts of water, which is absent of any energy, into the digestion system. The thickened slurry may also be subjected to pulping which reduces the size of the solid particles within the slurry to avoid clogging within the digestion system, even for substrate streams having high solid content. The pre-treatment system also removes sand as well as other inorganic, indigestible matter from the substrate.

During pre-injection treatment, the chemical characteristics of the biomass may also be adjusted as needed to achieve a desired pH level, carbon to nitrogen ratio, and surface tension. The weight ratio between carbon and nitrogen in organic matter for anaerobic digestion should be optimally 25:1 in order for the degradating bacteria to have sufficient nitrogen for their reproduction and the conversion of the carbon to an intermediate compound that can be converted to biogas. However, if the nitrogen content within the substrate stream is elevated, such as a carbon-nitrogen ratio of 5:1, the imbalance can lead to the production of larger amounts of ammonia, which can destroy the bacteria within the digestion system.

To reduce the surface tension of the biomass and thus reduce the likelihood of biomass clustering and floating layer formation in the digestion system, chemical salts may be added to the substrate prior to its injection to the digestion system. The reduced surface tension also reduces the amount of pumping energy required to inject the substrate to the digestion system.

In yet a further pre-treatment, additives are added to the biomass. The conversion of acetic acid to methane and carbon dioxide by a mixed methanogenic population in an anaerobic digester can be stimulated and enhanced by the addition of trace amounts of nickel, cobalt, and zinc. These substances can either be added to the substrate in the mix-tank, during pretreatment, injected directly into the multi-chambered digestion system or be built into the fixed biomedia so that it is slowly released to the substrate. The addition of nickel, cobalt, and zinc allows for the accumulation of a thicker methanogenic fixed film. This thicker biofilm can enable increased biogas production, the treatment of larger substrate volumes, and shortened reactor times.

In one form of the invention, the digestion system consists of a number of separate digestion chambers or vessels flow coupled in series with one another. The digestion system provides a sequential degradation of the substrate. Sequential degradation adds to the efficiency of the degradation process. More particularly, the active bacteria in each chamber only “sees” the substrate that was left unprocessed by the previous, upstream digestion chamber. As a result, the bacteria in each chamber can be balanced to specialize in converting the unconverted organics from the upstream chamber. Additionally, since each chamber is individually balanced, the unconverted substrate will be passed to a downstream chamber rather than converted undesirably to volatile fatty acids.

The number of chambers can vary depending on the amount and type of substrate to be processed. Each chamber or vessel has four compartments: a head space, a media space, a distribution space, and a sediment space. The head space is equipped with a gas outlet through which biogas may be collected from the chamber. The media space contains immobilization media for the degradation of the substrate and the formation of the biogas. The distribution space facilitates that passing of unconverted substrate to a downstream chamber, and the sediment space is used to collect any sediment that was not collected during pretreatment of the substrate.

In one form of the invention, the immobilization media includes a block of vertically oriented tubular structures. In another embodiment the immobilization media includes vertically oriented plates arranged in a way that vertically tubular structures are obtained. In one embodiment, the structures are roughed or scoured to increase surface area. In another embodiment, the tubular structures are formed from a metal or plastic mesh. The lateral openings formed in the mesh allow for cross-flow between structures and provide increased surface area for biofilm adhesion. The size and the number of tubular structures can vary and thus be optimized for a given substrate treatment application. Additionally, the shape of the media can be non-tubular, i.e., a non-circular cross-section. Further, the makeup of a given media section within a chamber can be non-uniform. In this regard, different diameter tubular structures, for example, may be used to form a given media section.

To facilitate degradation and biogas formation, the substrate is pumped to the most upstream chamber at a prescribed flow velocity, whereupon the substrate flows downward through the media section and interacts with bacteria formed along the walls of the tubular structures. Due to the design of the chambers that enables a plug flow the substrate will be pushed up through the media section in the next chamber and down again in the following chamber and so on, until the media has passed through all the chambers. This interaction causes the bacteria (“biofilm”) to breakdown the substrate and produce biogas. Biogas, and nutrient rich effluent, is thus produced and can be collected at each chamber. Substrate that is not captured by the biofilm is then passed to a downstream chamber for degradation and biogas formation. This process is then repeated through the multiple chambers of the digestion system. Each chamber may also be equipped with a recirculation pump to provide intrachamber recirculation to enhance degradation and biogas formation. A pump may also be used to circulate unconverted substrate from a downstream chamber to an upstream chamber. In one embodiment, sediment is removed automatically from the bottom of the chambers via a number of augers.

The present invention further provides an anaerobic digestion system in which two different kinds of microbes are used to form a biofilm for the degradation of the organic substrate and the production of biogas. The microbes generally consist of a “fast” bacteria group that degradate the complex organic substrate into acetic acid, alcohols, carbon dioxide and hydrogen gas, and a “slow” bacteria group that converts the degradated substrate to methane biogas. The degradating bacteria are robust, replicate approximately every twenty minutes under various conditions. The methane forming bacteria have a slow growth rate and need very stable temperature and pH conditions.

Once the bacteria have attached to the immobilization media within the various chambers, these bacteria “families” interact with the organic substrate as it passes through the chambers. According to one embodiment of the invention, the conditions within the chambers are setup so that the chemical selection targets equalizing of the production speed of the bacteria groups in a way that allows the process to occur at the speed of the fastest bacteria instead of allowing the slowest bacteria to be rate-limiting for the whole degradation and biogas formation process. In other words, the active biology within the chambers is balanced by controlling the way the different bacteria in the different chambers are fed.

Balancing the bacteria within the chamber is carried out by filling the chambers with substrate that is to be processed once and then to stop feeding substrate to the chambers. The content within the chambers is then heated and degradation and de-gassing takes place until no more gas is leaving the chambers. During this first stage of the balancing process, fatty acid content, pH level and other control parameters are measured in each chamber. Next, the “fast” bacteria, which are primarily responsible for degradation of the substrate, are allowed to sit inactively within the biofilm that is formed on the immobilization media without being fed “new” substrate to degradate. At the same time, the “slow” bacteria are fed in a controlled way that allow for that bacteria to produce biogas and replicate at its highest pace.

More particularly, the slow bacteria are fed selected metabolic products of the degradating bacteria. The metabolic products are initially fed at the rate of 1 gram per chamber volume liter per day. When the methane production occurs according to the dosed metabolic bacterial feed and the concentration of methane bacteria feed inside the chambers does not increase, the amount of metabolic product will be increased each day and ultimately terminated when the methane producing bacteria accepts the load of 25 to 45 gram of metabolic feed per chamber volume liter per day. As the balancing process is carried out, toward the end of the process, actual substrate rather than metabolic product will be fed to the chambers. When the concentration of the degradating bacteria is at an acceptable level within the chambers, the biological balancing process is complete.

The substrate treatment system of the present invention may also include a desulfurizer system for cleaning of the raw biogas that is produced during anaerobic digestion of organic substrate. The desulfurizer system can also be used to clean raw biogas that originates from landfills or wastewater treatment plants. The sulfur removal process of the present invention exposes the raw biogas to microorganisms, such as Thiobacillus denitrificans. Such microorganisms convert sulphide into free elementary sulfur in a moderately oxidized environment. The desulfurization system of the present invention includes a reactor containing media on which the sulfur-converting microorganisms live and grow. Raw biogas is fed to the reactor as is a countercurrent liquid stream which consists of digested slurry from the digestion process. The liquid stream keeps the media moist, supplies the microorganism on the media with feed, and inoculates the media with bacteria. The microorganisms living on the media interact with the biogas to convert the hydrogen sulphide to sulfur. The sulfur is washed down from the media by the countercurrent liquid stream.

It is therefore an object of the invention to provide an organic substrate treatment system that can be tailored, and adjusted as needed, to obtain optimized efficiency in terms of anaerobic degradation, retention time, and biogas yields whether for a single organic substrate, e.g., manure, or a composition of organic substrates, e.g., manure, whey, silage, etc.

It is another object of the invention to treat organic substrate prior to its anaerobic digestion to maximize its degradation and subsequent biogas formation during the digestion process.

It is another object of the invention to provide a substrate treatment system that can be used to process organic substrate having a dry matter content generally up to 15 percent and, in some cases, in excess of 15 percent.

It is another object of the invention to provide a substrate treatment system that can be implemented in various sized installations, such as hobby farms, corporate farms, milk producers, breweries, distilleries, centralized biogas plants, food producers, wastewater treatment facilities, and the like.

Therefore, in accordance with one aspect of the invention, an organic substrate treatment system is provided that breaks down organic substrate into a nutrient-rich effluent that can be used as fertilizer and produces biogas that can be used as heat energy or converted to electricity. The substrate treatment system may carry out a pre-treatment process in which the organic substrate is fluidized for easier injection into a digestion system. The pre-treatment of the organic substrate may also include the introduction of additives that enhance the degradation and biogas formation processes. The substrate treatment may include the sequential degradation of organic substrate using a number of digestion chambers interconnected in series. The biogas collected from one or more of the chambers is then fed to a cleaning system that removes sulfur from the biogas.

In accordance with another aspect of the invention, an anaerobic digestion system for use with a substrate treatment system is provided. The anaerobic digestion system includes a number of digestion chambers flow coupled in series to provide improved substrate degradation and biogas formation. Substrate that is not converted in a given digestion chamber is fed to downstream chamber for conversion. Segmenting the digestion process into multiple chambers allows each chamber to be biologically balanced to handle the biological makeup of the substrate that it is fed.

In accordance with another aspect of the invention, a media for the formation of biofilm is provided. The media includes a block of uprightly oriented tubes. The tubes are defined by an annular or square wall formed of any shape of mesh. In one embodiment, the tubes have a diamond shaped mesh.

In accordance with another aspect of the invention, a biogas desulfurizer system is provided. The desulfurizer system can be used to clean biogas formed from various biological processes.

Various other features, objects and advantages of the invention will be made apparent from the following description taken together with the drawings.

### BRIEF DESCRIPTION OF THE DRAWINGS

The drawings illustrate the best mode presently contemplated of carrying out the invention.

In the drawings:

FIG. 1 is a general schematic of a substrate treatment system according to the present invention;

FIG. 2 is a block diagram of the pre-treatment system of the substrate treatment system shown in FIG. 1;

FIG. 3 is a block diagram of the digestion system of the substrate treatment system shown in FIG. 1;

FIG. 4 is a schematic section view of a digester tank of the digestion system of FIG. 3;

FIG. 5 is an isometric view of a block of immobilization media usable with the digester tank shown in FIG. 4;

FIG. 6 is a section view of the block of immobilization media taken along line 6-6 of FIG. 5;

FIG. 7 is a block diagram of the biogas cleansing system of the substrate treatment system shown in FIG. 1; and

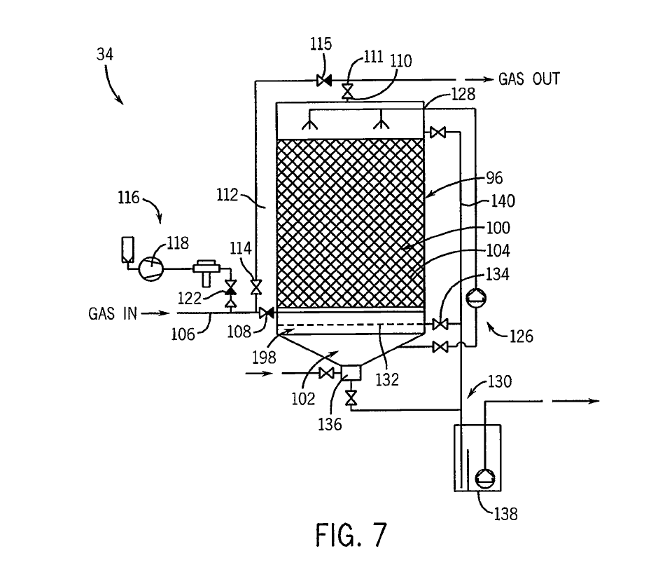

FIG. 8 is an isometric view of an immobilized media that may be impregnated with digestion enhancing agents according to another embodiment of the invention.

### DETAILED DESCRIPTION

The present invention will be described with respect to the treatment of animal and other organic substrate collected and treated onsite at a farm. It is understood however that the present invention could be used for the treatment of non-farm substrate or any kind of organic matter. Additionally, the invention could also be used for the offsite treatment of farm and other organic substrate. The description of the invention for the treatment of animal substrate shall in no way limit the scope of the appending claims.

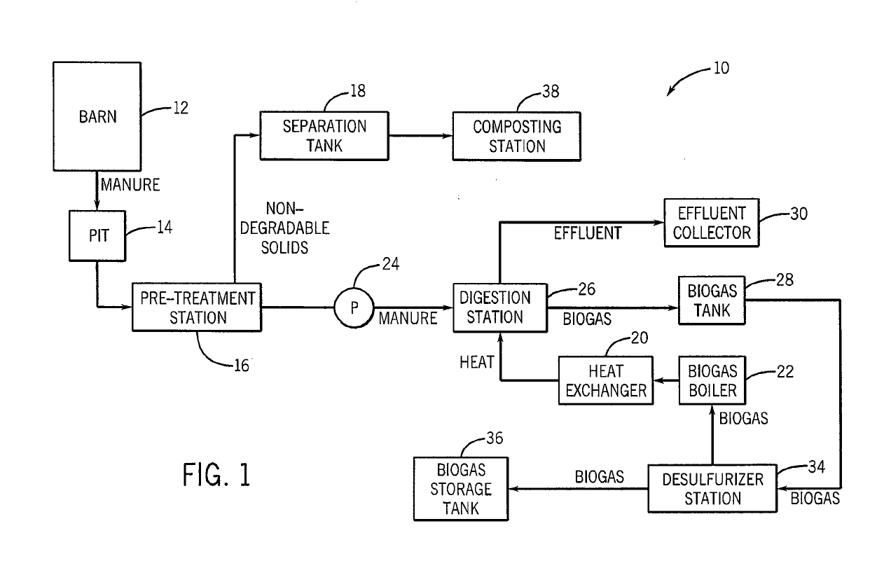

FIG. 1 is a block diagram of one implementation of a substrate treatment system 10 according to the present invention. In this implementation, raw manure is collected from a barn 12 and can be optionally stored in a collection pit or storage tank 14. The manure, either scraped directly from barn 12 or recollected from the collection pit/storage tank 14, is fed to a pretreatment station 16. The specifics of the pretreatment station 16 will be described in greater detail below, but in general, the pretreatment station 16 removes non-degradable (inert) solids from the manure, which are fed to a separation tank 18, thickens the raw manure into a fluidized slurry, and applies additives to the slurry to enhance the degradation and biogas formation process. The fluidization of the slurry generally involves heating of the raw manure. In this regard, the substrate treatment system 10 may include a heat exchanger 20. Biogas captured from the manure during anaerobic digestion may be used in a biogas boiler 22 to provide heated water to the heat exchanger. During startup or times of depleted biogas, propone or other gas source (not shown) may provide gas to the boiler 22.

The pretreated manure is then injected by pump 24 to a digestion station 26, which will be described in greater detail below. In general, the digestion station 26 includes immobilization media containing a biofilm of microbes that breaks down the manure into volatile fatty acids, alcohols, carbon dioxide, and hydrogen gas. Other microbes contained within the biofilm convert these products into biogas, which can be captured from the digestion station 26. At the digestion station 26, anaerobic digestion of the manure takes place to yield the aforementioned biogas, e.g., methane, and effluent that can be subsequently processed and used as fertilizer. In this regard, the substrate treatment system 10 further has biogas collector 28 and an effluent collector 30, to which effluent is fed from the digestion station 26 by one or more pumps 32.

The present invention also contemplates that the biogas can be converted to electrical energy. As the raw biogas will generally have an elevated sulfur content, the substrate treatment system 10 further has a desulfurizer station 34 that process the raw biogas to remove, or otherwise reduce its sulfur content. The specifics of the desulfurizer will be described in greater detail below. The biogas, after having its sulfur content reduced by the desulfurizer station 34, is then fed to a storage tank 36, where it can subsequently sent to a suitable conversion station (not shown) for conversion to electricity, which may be on or offsite.

It will be appreciated that the organic solids fed to the separation tank 18 may fed to a composting station 38 for compositing in a conventional manner. Inorganic solids fed to the separation tank 18 may be collected in a known manner and disposed of accordingly, e.g., collected by a refuse collection company and transported to a recycling center or landfill.

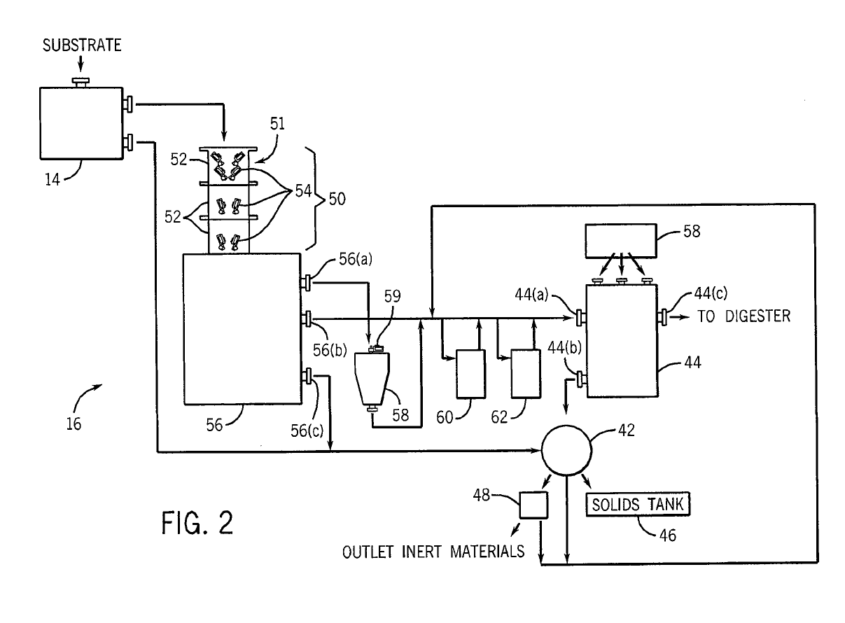

Turning now to FIG. 2, the pretreatment station 16, as described above, treats the raw manure before its injection to the digestion station 26. The pretreatment station 16 is designed to maximize the production of biogas during anaerobic digestion. In this regard, it is contemplated that the pretreatment station 16 is an optional, but certainly preferred, component of the overall substrate treatment system 10. The pretreatment station 16 is configured to ensure that the raw organic material, regardless of origin, can be processed in the anaerobic digestion station 16 in such a way that the bound energy and the bound nutrients in the organic material are released optimally.

The pretreatment station 16 carries out multiples processes to prep the raw manure for anaerobic digestion. In a first process, the raw manure is fed to a separation tank (“phase separation unit”) 40 that separates the raw manure into two layered phases over time based on the density of the components within the manure. The heavy materials, e.g., solids, accumulate at the bottom of the separation tank 40. These “dense” materials are transferred from the bottom of the tank 40 to a central separation unit (“liquid-solid separator”) 42, which separates the dense materials from its carrier liquid. The liquid is pumped to a mix tank 44 whereas the solids are passed to a solids collection tank 46 for disposal. It should be noted, as will be described in greater detail below, depending upon the type of waste, the liquid output of the liquid-solid separator may be input to a pasteurization station and/or sterilization station prior to its injection into the mix tank 44. It should further be noted that if the solids include sand, such as from a sand bedded dairy farm, the sand is further processed for reuse in a sand washer 48. The principal reason for removing the components that have a density greater than one is these inert materials have no value with respect to energy or as fertilizer nutrients. Furthermore, such materials may wear down pumps and other rotating equipment. The materials could also otherwise be collected as sediment, which require stoppage of the digestion process for its removal.

The lighter or less dense components float to the top of the separation tank 40 and are transferred to a downsizer assembly 50. The downsizer assembly 50 includes a downsizing unit 51 having a housing 52 containing several pairs of rollers 54. Each roll has its own cutting and/or grinding profile and each pair cooperate to grind and otherwise downsize the components to a puree in which the fibrous structure is completely broken down. In one embodiment, the rolls in each pair rotate with differential speed in proportion to another, and the speeds can be variably controlled. The grinding profiles and the rotational speeds of the rollers are set and controlled to optimize grinding for a given organic material. Variably controlling the speed of the rollers is done in a conventional manner. In one preferred embodiment, the downsizer assembly 50 consists of modules of individual downsizing units 51 with each unit having one or more pairs of rollers 54. The number of modules and the profiles of the rollers can thus be tailored to meet the demands of a given substrate. Preferably, the modules are stacked vertically so that the substrate flows down through the modules under the force of gravity. Alternately, pump(s) or other mechanical devices could force the substrate through the modules.

From the collection tank 56, floating substrates in the organic material is fed to a pulping unit 58. More particularly, the upper end of the collection tank 56 has an outlet 56(a) that is flow coupled to an inlet of the pulping unit 58. In one embodiment, the floating substrates are pumped to the pulping unit 58 by pump 59. The pulping unit 58 includes a rotating cutting knife (not shown) through which the organic material is passed and any remaining fragments in the organic material are cut and ripped apart. Rotational speed of the knife and exposure time of the organic material to the knife can be adjusted as needed to meet the specifics of a given organic material. It will be appreciated that the grinding the raw manure or biomass to a puree is to give optimum access for the bacteria during digestion, which leads to higher degradation rates and methane formation rates. Further, the load on pumps used to pump the organic material is reduced.

The components that are suspended within the substrate in the collection tank 56, i.e., have a density at or near one, are fed to the mix tank 44. In this regard, the collection tank 56 has an outlet 56(b) generally positioned at a mid-height of the collection tank 56. In effect, these components are suspended in the liquid that is output by the downsizer assembly 50.

The solid components within the substrate contained in the collection tank 56 float generally to the bottom of the collection tank 56. In this regard, the lower end of the collection tank 56 has an outlet 56(c) that is flow coupled to the liquid-solid separator 42, where the solids are processed as described above.

The conversion of acetic acid to methane and carbon dioxide by a mixed methanogenic population in an aerobic digester is stimulated and enhanced by the addition of nickel, cobalt, and zinc in trace amounts to the puree contained within the mix tank 56 by a dosing unit 58. The addition of trace elements of nickel, cobalt, and zinc enhances digester performance. Biogas production can be increased substantially by the addition of these additives. Moreover, these additives allow for greater volumes of substrate to be treated and reactor (“digester”) residence time to be reduced. The addition of nickel, cobalt, and zinc enable the accumulation of a thicker methanogenic fixed film within the digestion system 26.

In addition to adding nickel, cobalt, and zinc, the dosing unit 58 may also add chemical salts to the organic material to reduce the surface tension of the material. Reducing the surface tension provides a number of benefits. For example, reducing the surface tension makes it easier to pump the material. Reducing the surface tension also reduces the likelihood of organic material clustering within the digestion station 26.

The dosing unit 58 may also add compounds to the organic material to adjust the pH level of the material. The methane forming bacteria contained within the digestion station 26 are sensitive to changes in their living conditions. As a result, the dosing unit 58 can add base or acidic components to the organic material, as needed, to achieve a desired pH level of the organic material.

The carbon to nitrogen ration within the organic material is also an important parameter of the digestion process. In this regards, carbon and nitrogen readings are preferably taken of the organic material within the mix tank 44. An ideal carbon to nitrogen ratio is about 25:1. In this regard, the dosing unit 58 may also add carbon or nitrogen to the organic material to achieve a desired carbon to nitrogen ratio. If the ratio is too low, e.g., 5:1, the high percentage of nitrogen can lead to the production of larger amounts of ammonia, which can be harmful to the living microorganisms within the digestion station 26.

In an alternate embodiment, the mix tank 44 may contain plate-like members (not shown) impregnated with nickel, cobalt, and zinc. In this alternate embodiment, the nickel, cobalt, and zinc are absorbed by the substrate as it is mixed within the mix tank 44. It is contemplated that the dosing unit 58 could also be used with this embodiment to inject supplemental amounts of nickel, cobalt, and zinc as needed.

In addition to the treatment of animal waste, e.g., manure, the substrate treatment system 10 may also provide anaerobic digestion of other animal byproducts. Animal byproducts are classified by the European Commission Regulation No. 1774/2002 into three categories. Category 1 byproducts are required by law to be incinerated or disposed in a landfill and cannot be used for the production of biogas and fertilizer. Category 2 byproducts include those animal byproducts that present a risk of contamination with other animal diseases, e.g., animals which die on farm or are killed in the context of disease control measures on farm or at risk of residues of veterinary drugs. These byproducts may not be used for feed, but may be used for biogas formation with appropriate processing. Category 3 materials are those derived from healthy animals slaughtered for human consumption and may be used in the production of feeds, fertilizer, and biogas with appropriate treatment.

The substrate treatment system 10, in addition to treating manure, may also be used to treat category 2 and category 3 products. Accordingly, the pretreatment station 16 includes a pressure sterilization unit 60 for pretreatment of category 2 materials. The pressure sterilization unit 60 is kept at a minimum temperature of 133° C. and at an absolute pressure of 3 bar. To achieve proper sterilization, a minimum sterilization period of 20 minutes is generally required. Alternately, or in addition, the pretreatment station 16 includes a pasteurization unit 62 for treatment of category 3 materials. The temperature of the material fed to the pasteurization unit 62 must be at least 70° C. and the pasteurization process generally requires at least one hour.

Both the pressure sterilization unit 60 and the pasteurization unit 62 have maximum particle size requirements to be effective. In this regard, the category 2 and the category 3 products are downsized using a downsizing assembly. The category 2 and category 3 products may be processed by downsizer assembly 50 or separate downsizer units (not shown).

The mix tank 44 in a substrate inlet 44(a) through which the substrate is fed from the collection tank 56 (and the sterilization unit 60 and the pasteurization unit 62). The mix tank includes one or more recirculation pumps (not shown) that circulates the substrate within the tank 44 to promote mixing with the chemicals that are added by the dosing unit 58 or contained on media within the tank 44. Solids are collected at the bottom of the mix tank 44 and are removed from the mix tank via outlet 44(b) and fed to the liquid-solid separator 42. The mixed substrate is passed via outlet 44(c) to the digestion station 26.

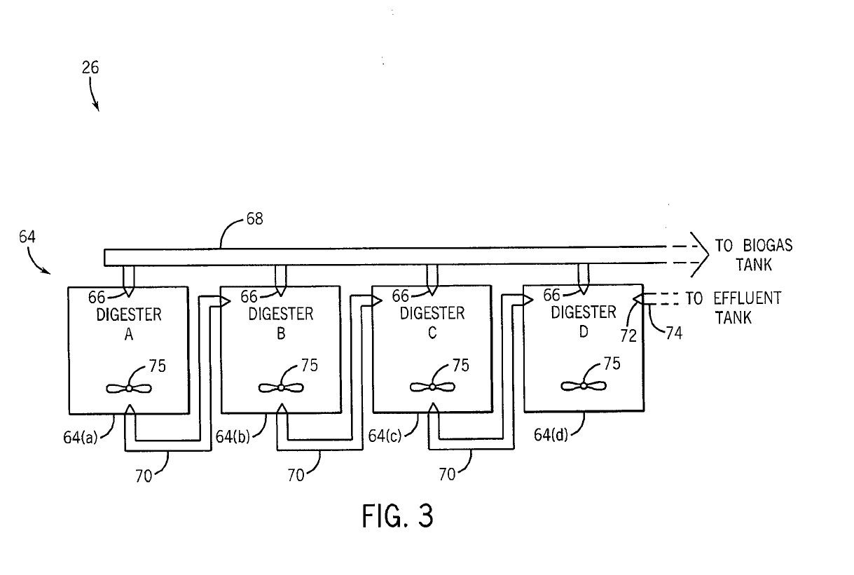

At the completion of the pretreatment or unification process, the slurry of organic material is prepped for anaerobic digestion at digestion station 26. Turning now to FIG. 3, the digestion station 26 includes a number of digester tanks 64 connected in series. The number of digester or reactor tanks 64 can vary depending on the type and amount of organic material to be processed. For some implementations, it is contemplated that a single, larger digester tank could be used rather than one or more modular digester tanks. The organic material is injected to the first digester tank 64(a) by pump 24 and this action, as it is continued, as well as the internal design of the digester tank that enables plug flow forces the organic material through the remaining tanks 64(b), 64(c), and 64(d). Solids contained within the substrate are passed through the chambers and ultimately fed back to the liquid-solids separator 42, FIG. 2.

In each digester tank 64, anaerobic digestion is carried out on the organic material to provide a sequential degradation of the organic material. In this regard, each tank 64 includes immobilization media containing a biofilm that breaks down the organic substrate to “degradators” which are in turn converted to methane gas. Sequential degradation provides a number of advantages over the single-reactor degradation of conventional substrate treatment systems. For example, by using sequential degradation, the biofilm within each digester tank sees only the organic material or biomass that was unprocessed by its upstream digester tank. As such, digester tank 64(c) sees only the biomass that is unprocessed by tanks 64(a) and 64(b). Because of the biofilm within each digester tank sees only the unprocessed material from its upstream tank(s), the biofilm in each chamber can be balanced so as to specialize in converting the organics that were left unconverted by the biofilm in the upstream tank. In short, the biofilm in a given tank does not have to process the all of the organics fed to the digestion station 26; it only has to process the biomass that it sees and, for that matter, only a portion of it as the unprocessed organics will be handled by the biofilm in a downstream tank.

In addition, by “specializing” the biofilm in each digester tank, it is difficult for the substrate treatment process to yield undesirable levels of volatile fatty acids that could otherwise overwhelm the digestion process.

As shown in FIG. 3, each digester tank 64 may be equipped a biogas outlet 66 that is flow coupled to a pipe 68. In another embodiment, only the most downstream digester tank, e.g., digester tank 64(d) has a biogas outlet. Biogas is captured f fed to biogas tank 22 for subsequent processing. Alternately, the biogas pipe 68 may be coupled to the desulfurizer station 34 so that the collected biogas is desulfurized before it is stored. It is also contemplated that biogas, once processed by the desulfurizer station 34, could be provided to the biogas boiler 22 for the generation of heat energy that can be used for temperature control within the digester tanks 64.

The digester tanks 64 are flow coupled to one another. In one embodiment, hoses 70 are used to interconnect the tanks 64; although, other types of flow couplings could be used. For example, in another embodiment, the digester tanks are welded together to form a single closed system and pathways, such as slits, are formed in the inner walls of the tanks to facilitate flow between adjacent tanks. The organic material is captured near the bottom of an upstream tank and is fed near the top of the downstream tank 64. The most downstream tank, e.g., tank 64(d), has an effluent outlet 72 to which conduit 74 is coupled. Conduit 74 passes the effluent from the tanks to the effluent collector 30, FIG. 1. Although not required, it is also contemplated that the effluent could be re-circulated back to the most upstream tank, e.g., tank 64(a), for additional biogas formation. Although not required, it also contemplated that organic material could be re-circulated within each tank by respective reciprocating pumps 75. Each digester tank 64 is independently balanced and temperature controlled. The tanks are heated initially by heat from an outside heat source but eventually are heated by biogas captured from the organic material. The tanks are preferably operated a constant temperature depending on the type of organic material to be processed, but the temperature of each tank is independently controlled. In one preferred embodiment, the tanks are held at a relatively constant temperature of 37° C. In another embodiment, the tanks are held at a relatively constant temperature of 53° C.

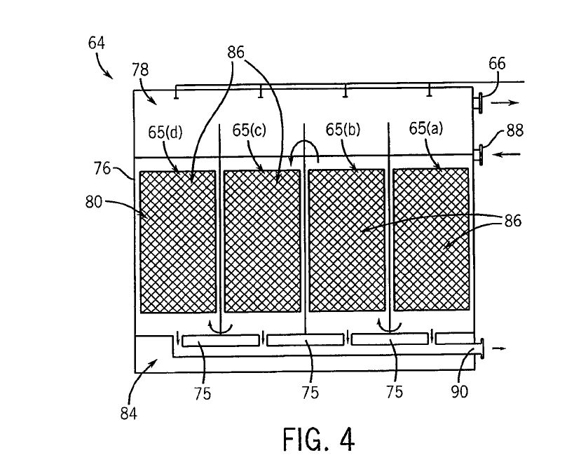

Referring now to FIG. 4, each digester tank 64 has a closed vessel 76 segmented into a head space 78, a media space 80, a distribution space 82, and a sediment space 84. The media space 80 contains multiple chambers 65(a)-(d), each containing a media (“bio-block”) 86, which will be described in greater detail below. The head space 78 is defined at the top of the vessel and contains an inlet 88 and the aforementioned biogas outlet 66. The distribution space 82 sits beneath the media space 80 and includes a fluid outlet (not shown) through which organic material can escape the vessel 76 and passed to a downstream vessel via a hose 70. In one embodiment, any sediment not captured during the pretreatment processes is manually scraped from the vessel 76. Alternately, an automatic discharge system could be used to remove solids and/or sediment from the bottom of the digester tank 64 through outlet 90. For example, the automatic discharge system could be operated to continuously remove solids from the bottom of the digester tank or could be operated such that outlet 90 is only open at certain instances to remove solids at predefined intervals, such as twice a day. The size of the vessel can vary depending on the type and volume of organic material to be processed. Preferably, the vessels have a diameter to height ratio of 1:2.

In one embodiment, the substrate is fed to the digester tank 64 via inlet 88 which is situated at or near the top of the digester tank 64. Under the force of gravity, the substrate flows toward the bottom of the tank through the media 86, which as described below, causes anaerobic digestion to take place for some of the substrate that is passed through the media 86. When the substrate reaches the bottom of the tank 64, a pump 75 or similar mechanical device forces the substrate upward back through media contained in the adjacent chamber. When the substrate reaches the top of the adjacent chamber, e.g., chamber 65(b), the substrate will overflow into one or more of the adjacent chambers, e.g., 65(a) and 65(c), whereupon the substrate will flow under the force of gravity down through the media contained within the chamber. It contemplated that portions of the substrate that have passed through the media contained in chamber 65(a) and pushed through the media contained in chamber 65(b) may fall back into chamber 65(a). It is also contemplated however that the digester tank 64 may be constructed to prevent substrate flow back to an upstream chamber. In one embodiment, each chamber is in direct fluid communication with the outlet 90. In another embodiment, only the most downstream chamber, e.g., chamber 65(d), is in fluid communication with the outlet 90.

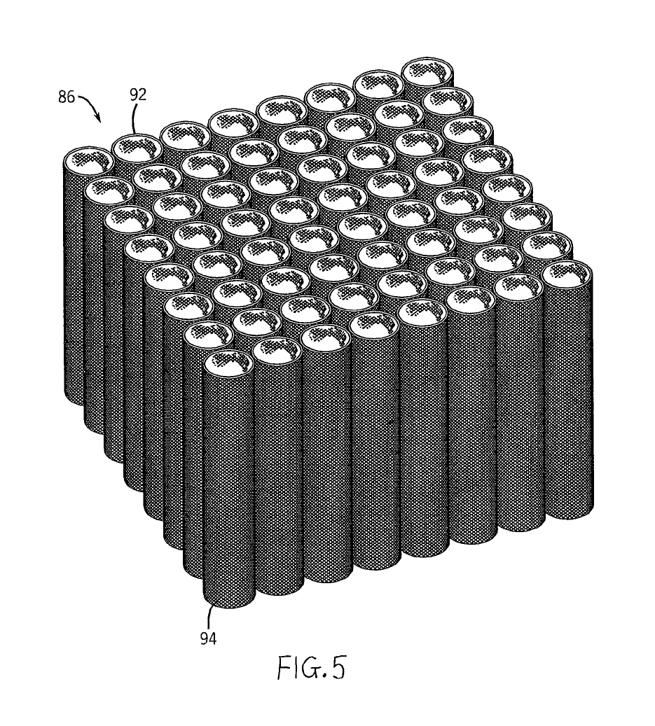

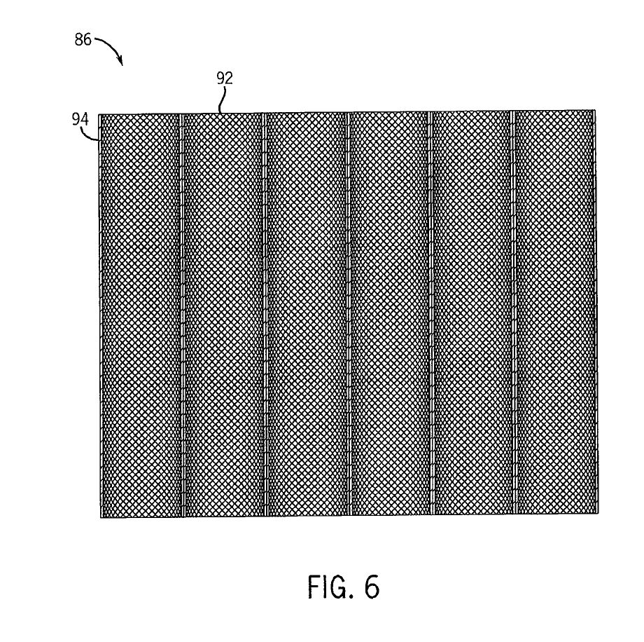

The bio-block 86 is fixedly anchored within the vessel 76. While different types and shapes of media for the fastening of biofilm may be used, in a preferred embodiment, the bio-block 86 includes an array of tubes 92, as best shown in FIGS. 5 and 6. The number of tubes 92 can vary depending on the type and volume of organic material to be digested. Further, while the illustrated bio-block 86 has tubes of uniform diameter and length, it is understood that the bio-block 86 may contain tubes of varying diameters and lengths. Moreover, the shape of the tubes could be different from those illustrated in the figures. For example, the “tubes” could have a square, rectangle, or oval cross-section. In a further example, the “tubes” could be helical in shape.

The material composition of the tubes 92 can vary, but preferably the outer walls 94 of the tubes 92 have a mesh pattern or are scoured. The mesh, which in one preferred embodiment, is a diamond mesh, or the scouring increases the surface area of the tubes 92 and enhances biofilm adhesion. Additionally, the mesh defines openings that allow for cross-flow between tubes which improves biofilm creation and the degradation process. In one embodiment, the tubes are formed from polyethylene but other materials may be used.

The bio-block 86 provides a modular media that can be stacked horizontally or vertically within the vessel 76 as needed to meet the demands of a given substrate treatment system. The modularity of the bio-block 86 allows multiple blocks to be used to satisfy the size of the vessel, which may vary from treatment site to treatment site.

The bio-block 86 provides an anchoring surface for the microorganisms that degrade the organic material and convert the “degradators” to biogas. In this regard, microbes anchor to the tubes 92 using cell adhesion and form families that create a permeable slime layer—a biofilm. This biofilm is an active biology that is ready to degrade and convert the organic material as the organic material is passed through the tubes 92. Thus, in a preferred embodiment, the tubes 92 are stacked vertically, but it is contemplated that other orientations may be used. Furthermore, the bio-blocks may be made out of a material which contains nickel, cobalt, and zinc that is slowly released to the substrate.

As described above, each digester tank 64 sees only the organic material that was not processed by its upstream tanks. In this regard, each tank 64 must be balanced and controlled independently of the other tanks based on the organic material that it sees. The manner in which each tank is balanced and controlled is generally the same for each tank.

For the digestion process to be most effective, there must be a balanced concentration between the bacteria that degrade the organic material and the bacteria that produce the biogas. The hydrolyzing bacteria that degrade organic material to fatty acids and other metabolites can multiply very quickly, e.g., 20 minutes. On the other hand, the bacteria that under anaerobic conditions utilize the residual products from these hydrolyzing bacteria multiply very slowly, e.g., 12-14 days, and are much more sensitive to changes in their living conditions, e.g., temperature and pH level. If the anaerobic digestion process is initiated with larger numbers of the “fast” bacteria and smaller numbers of “slow” bacteria, the conversion rate will be limited by the number of slow-growing bacteria. While this is a natural balance, it is an imbalance for optimized biogas production. The present invention utilizes immobilized bacteria and as a result the balance or ratio between the “fast” and “slow” bacteria can be changed to increase the ratio of “slow” bacteria to enhance biogas production. Moreover, once this preferred balance is obtained, it can be maintained provided the organic material remains generally unchanged and/or the operating conditions, e.g., temperature and pH levels, remain generally unchanged.

Accordingly, the present invention further includes a method for achieving a proper balance within each of the digester tanks. Balancing the bacteria within the tanks is carried out by filling the tanks with substrate that is to be processed once and then to stop feeding substrate to the tanks. The content within the tanks is then heated and degradation and de-gassing takes place until no more gas is leaving the tanks. During this first stage of the balancing process, fatty acid content, pH level and other control parameters are measured in each tank. Next, the “fast” bacteria, which are primarily responsible for degradation of the substrate, are allowed to sit inactively within the biofilm that is formed on the immobilization media without being fed “new” substrate to degradate. At the same time, the “slow” bacteria are fed in a controlled way that allow for that bacteria to produce biogas and replicate at its highest pace.

More particularly, the slow bacteria are fed selected metabolic products of the degradating bacteria. The metabolic products are initially fed at the rate of 1 gram per tank volume liter per day. When the methane production occurs according to the dosed metabolic bacterial feed and the concentration of methane bacteria feed inside the tanks does not increase, the amount of metabolic product will be increased each day and ultimately terminated when the methane producing bacteria accepts the load of 25 to 45 gram of metabolic feed per tank volume liter per day. As the balancing process is carried out, toward the end of the process, actual substrate rather than metabolic product will be fed to the tanks. When the concentration of the degradating bacteria is at an acceptable level within the tanks, the biological balancing process is complete.

As noted above, the substrate treatment system 10 may also be equipped with a biogas cleansing or desulfurizer station 34. The desulfurizer station carries out a biological fixed film and wet countercurrent scrubber process for cleaning the raw biogas. The desulfurizer station and process of the present invention can be used with agricultural or industrial digestion process or also used to clean raw biogas that originates from landfill sites or from a wastewater treatment plant, for example. The desulfurizer station 34 is capable of providing at least a twenty-five fold reduction in hydrogen sulphide content and reduces siloxanes content to less than 15 ppm.

The desulfurizer station 34 is equipped with a reactor tank 96 that is preferably square or round and is fabricated from stainless steel, fiber glass, or coated mild steel. The operating pressure of the tank is between −20 mbar and 40 mbar. The tank 96 generally has a head space 98, a packed column space 100, and a sump space 102. The tank 96 is insulated with 150-200 mm of glass or rock wool and is preferably protected from weather by cladding.

The packed column space 100 is loaded with packing media 104 preferably having a surface area of 200 m² per m³. The ratio of packed media 104 to biogas to be treated is 1 m³ of packed media to 8 m³ of biogas/hour. The packed media can take many forms and in one embodiment is similar to the media contained within the digester reactor tank 64. It should be noted however that while the media contained within the reactor tank 64 may be impregnated or otherwise contain nickel, cobalt, and zinc, the packing media 104 is not impregnated with such chemicals.

The raw biogas is fed to the reactor tank 96 via an inlet 106 at the bottom of the reactor tank 96. In a preferred embodiment, the inlet 106 is fitted with a non-return valve 108. The top of the tank 96 is fitted with an outlet 110 and valve 111 that is in fluid communication with the head space 98. A bypass pipe 112 and valves 114, 115 are also provided between the gas inlet 106 and the gas outlet 110.

The desulfurizer station 34 has an air system 116 to supply atmospheric air to the gas stream. In a preferred embodiment, the amount of atmospheric air is 1 to 4 percent of the biogas volume. The air is injected by a blower or a compressor 118, which is frequency controlled. Preferably, the air is injected into the gas inlet 106 with the flow of air into the gas stream controlled by a non-return magnetic valve 122. At the gas outlet 110, there is an oxygen sensor (not shown) that measure the oxygen and provides feedback to the control system (not shown) for the air system 116.

The desulfurizer station 34 also has a sprinkling system 126 that adds digested slurry to the biogas. The slurry is injected at the top of the reactor tank 96 via inlet 128 and, in this regard, is counter-injected to the flow of biogas. The slurry contains nutritive salts and bacteria and maintains the pH level within the reactor tank 96 within a desired range. The sprinkling system 126 also keeps the packed media moist which is needed for effective biogas cleansing. In one embodiment, the sprinkling system 126 injects the digested slurry at predefined intervals, e.g., ten minutes, rather than a continuous spray.

A heat system 130 keeps the reactor tank at a preferred temperature of 30° C. The heating is provided by pipe coil 132 contained at the bottom of the reactor tank 96. Heat is provided by circulating heated fluid, e.g., water, through the pipe coil at a maximum temperature of 60° C. At higher temperatures, the fiber content within the slurry will burn onto the pipe coil thereby reducing heat transfer. A thermo-controlled regulating valve 134 is provided to control the flow of fluid through the pipe coil 132.

As noted above, slurry is counter-injected to the reactor tank 96. As such, a slurry sump 136 is provided that is flow coupled to the sprinkling system 126. Preferably, the slurry sump 136 contains approximately one percent of the amount of biogas to be treated per hour. For example, if 350 m³ of biogas it be treated per hour, the sump content should be 3.5 m³. The slurry is preferably digestate—degassed manure—from the anaerobic digestion process.

The air system 116 may also be used to clean the reactor tank 96 at prescribed intervals. After the reactor tank 96 has been trained of slurry and any residual biogas, the reactor tank is filled with lukewarm water from a water source (not shown). The air system 116 then pumps air into the tank 96, which causes the packing media to be lifted and agitated. This causes the thick biofilm on the media to come loose and separate from the media. The separated biofilm may then be flushed from the tank 96 as the lukewarm water is drained. Fresh slurry may be supplied to the rector tank 96 as biogas injection is also resumed. The remnants captured form the reactor tank 96 could be used as a sulfur supplement for fertilizer.

The desulfurizer station 34 further includes a condenser 138 that removes water from the biogas after it leaves the reactor tank 96. The condenser 138 includes a conduit 140 that is in fluid communication with the head space 98 of the tank 96. A portion of the conduit or pipe 140 is preferably buried in the ground so that water in the biogas is condensed at a dew point corresponding to the temperature in the ground. As the biogas is passed through the condenser, the water will be condensed thereby reducing the water content of the biogas. The condensed biogas is then fed to a storage tank, e.g., collector 28.

It will be appreciated that the operating parameters of the desulfurizer station 34 may vary but in a preferred embodiment, the station 34 is operated at an operating temperature of approximately 30° C. and at a pH level between 6.5 and 7.5. Gas flow is kept between 0.25 to 1.0 meters per second, and biogas retention time in the reactor time is a minimum of three minutes.

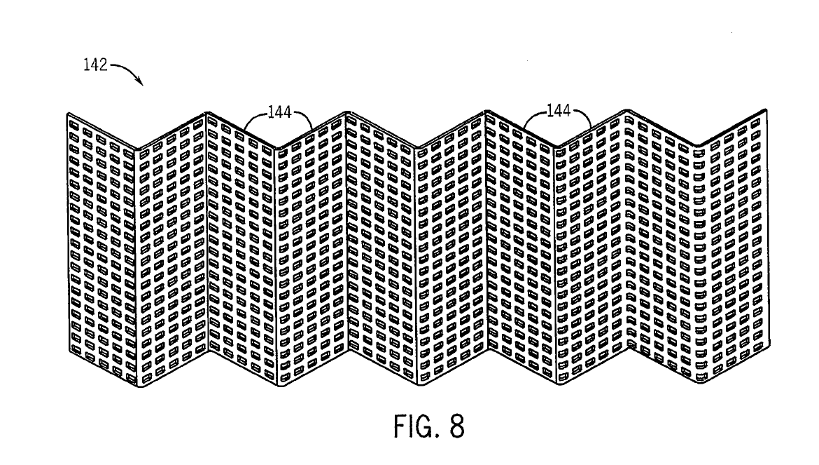

As referenced above, the immobilization media may take many forms. In one embodiment, which is illustrated in FIG. 5, the immobilization media is in the form of a set of tubular structures 86 joined together as a single unitary structure, or bio-block. FIG. 8 illustrates another immobilization media according to a further embodiment of the invention. In this embodiment, the immobilization media is the form of plate 142 defined by a series of planar sections 144 with adjacent section angled with respect to one another so that collectively the plate 142 has a wave-like orientation. Each planar section 144 is formed of a mesh material, or otherwise includes crevices, openings, holes, and the like for cell adhesion, as described above with respect to FIG. 5. The number of plates 142 within a given digester chamber and the arrangement of the plate(s) can vary depending upon the type and amount of substrate to be processed. It will be appreciated that the shape and number of crevices formed in the planar sections can vary. Moreover, it is understood that the plate may be formed from a set of non-planar sections. Further, it is understood that the plate may be impregnated with various chemical agents, such as nickel, cobalt, and zinc, to enhance or otherwise promote anaerobic digestion of the substrate. Impregnating the media may speed up achieving a biological balance within the digester tanks when compared to conventional fixed film media.

It will therefore be appreciated that the present invention provides a substantially complete treatment of organic substrate to biogas and nutrient rich effluent. The invention can be used to produce biogas from animal waste, but may also be used to produce biogas from crop residue, whey, sewage, wastewater, and the like. The invention may be scaled to meet the demands of different plants that produce organic substrate, including corporate farms, hobby farms, milk producers, cheese makers, breweries, municipal wastewater treatment plants, landfills, distilleries, and the like. Further, it is contemplated that after initial start up, the substrate treatment system 10 can be used to produce more energy than is required to carry out the anaerobic digestion and the biogas desulfurizing. As such, the invention can process organic substrate to provide an energy surplus that can be used to power onsite machinery or sold offsite as a secondary revenue source.

Various alternatives and embodiments are contemplated as being within the scope of the following claims particularly pointing out and distinctly claiming the subject matter regarded as the invention.

1. A system for treatment of organic substrates, comprising:
a pretreatment station that conditions organic substrate for anaerobic digestion;
a digestion station flow coupled to the pretreatment station and that anaerobically digests the organic substrate into biogas and effluent; and
a desulfurizer station flow coupled to the digestion station that reduces sulfur content within the biogas output by the digestion station.

2. The substrate treatment system of claim 1 wherein the digestion station includes a number of tubular structures containing bacteria that anaerobically digest the organic substrate.

3. The substrate treatment system of claim 2 wherein the number of tubular structures includes vertically oriented tubes having walls formed of a meshed material.

4. The substrate treatment system of claim 3 wherein the meshed material has a mesh pattern.

5. The substrate treatment system of claim 1 wherein the pretreatment station includes a dosing unit that adds digestion enhancers to the organic substrate prior to its anaerobic digestion.

6. The organic substrate treatment system of claim 5 wherein the digestion enhancers include at least one of nickel, cobalt, and zinc.

7. The organic substrate treatment system of claim 1 wherein the digestion station includes a number of reactor tanks interconnected in series, each of the tanks containing at least one block of immobilization media supporting a biofilm, and wherein the number of reactor tanks provide sequential degradation of the organic substrate.

8. The organic substrate treatment system of claim 1 wherein the desulfurizer station includes a reactor tank containing bacteria that interact with biogas supplied thereto to convert sulfur dioxide in the biogas to sulfur.

9. The organic substrate treatment system of claim 1 wherein the pretreatment station includes a grinding assembly configured to cut fibrous material contained within the organic substrate.

10. The organic substrate treatment system of claim 1 wherein the digestion station is configured to convert organic substrate to biogas in less than four days.

11. A biogas production apparatus for producing biogas from the anaerobic digestion of organic substrate, comprising:
a dosing unit that add additives to the organic substrate to enhance the anaerobic digestion of the organic substrate;
a number of biogas plants interconnected that carry out sequential degradation of a plug of organic material to provide raw biogas; and
a biogas cleansing unit to which the raw biogas from the biogas plants is fed, wherein the biogas cleansing unit provides a decrease in sulfur dioxide content of the raw biogas.

12. The apparatus of claim 11 wherein the biogas cleansing unit includes a reactor containing a packed media supporting microorganisms that convert sulfur dioxide in the raw biogas to elementary sulfur.

13. The apparatus of claim 12 wherein the dosing unit adds at least one of nickel, cobalt, and zinc to the organic substrate.

14. The apparatus of claim 11 wherein each biogas plant contains a bio-block of immobilized media containing bacteria that convert organic substrate to biogas and effluent, and wherein each bio-block includes a set of vertically oriented members formed of diamond mesh material.

15. The apparatus of claim 14 wherein the members have an annular cross-section and further have a uniform diameter.

16. The apparatus of claim 11 wherein the dosing unit further adds chemical salts to the organic substrate to reduce surface tension of the organic substrate.

17. The apparatus of claim 11 further comprising at least one of a pressure sterilization unit for pre-digestion treatment of category 2 organics and a pasteurization unit for pre-digestion treatment of category 8 organics.

18. A method of biogas production from organic material, comprising:
thickening the organic material into a fluidized slurry;
adding at least one of nickel, cobalt, and zinc to the fluidized slurry;
adding chemical salts to the fluidized slurry for reducing the surface tension of the fluidized slurry;
passing the fluidized slurry through a filter containing microorganisms that interact with the fluidized slurry to form at least volatile fatty acids;
converting the volatile fatty acids to biogas; and
capturing the biogas.

19. The method of claim 18 wherein the passing the fluidized slurry includes cascading the fluidized slurry through a number of filter connected in series.

20. The method of claim 18 further comprising reducing sulfur dioxide levels in the biogas.
